(19) United States
(12) Patent Application Publication
(10) Pub. No.: US 2019/0381262 A1
(43) Pub. Date: Dec. 19, 2019

(54) A DEVICE FOR INCREASING THE EFFICACY OF A METERED DOSE INHALER

(71) Applicants: Jonny COPPEL, Esher (GB); George NASH, London (GB)

(72) Inventors: Jonny COPPEL, Esher (GB); George NASH, London (GB)

(21) Appl. No.: 16/466,475

(22) PCT Filed: Dec. 8, 2017
(86) PCT No.: PCT/GB2017/053693

§ 371 Date: Jun. 4, 2019

(30) Foreign Application Priority Data
Dec. 8, 2016 (GB) 1620920.7

(51) Int. Cl. A61M 15/00; B33Y 80/00

(52) U.S. Cl. CPC A61M 15/0086; A61M 15/009; A61M 15/0021; A61M 15/0016; A61M 2205/586; B33Y 80/00; A61M 2205/3584; A61M 2205/3334; A61M 15/0018

(57) ABSTRACT

A closure (100) for a container (6) is disclosed. The closure comprises a fixture (11) for attaching the closure to the container, a medicament port (14) for receiving a medicament dispenser (2) and discharging a medicament from the dispenser within an internal volume of the container, an air inlet port (5) for drawing air into the container, a mouthpiece (3) and a valve (4). The valve has an open position which permits air to be drawn from the internal volume of the container into the mouthpiece when a user inhales, and a closed position which seals the mouthpiece from the container and permits air exhaled by the user into the mouthpiece to be vented to atmosphere.

### FIELD OF THE INVENTION

The present invention relates to a device for increasing the efficacy of metered dose inhaler. In particular the present invention provides an alternative to conventional spacers for metered dose inhalers.

### BACKGROUND TO THE INVENTION

Asthma is a condition that affects the lungs and is characterised by reversible narrowing or tightening of the airways in the lungs. Worldwide, it is estimated that up to 334 million people suffer from asthma. The majority of asthma sufferers are prescribed reliever meter dosed inhalers (MDIs). These inhalers contain medication that opens up (bronchodilates) airways in the lungs during acute exacerbations of asthma (“asthma attacks”). The efficacy of these inhalers is greatly enhanced by the use of a spacer. A spacer is typically a tube or a cylinder that is attached to the metered dose inhaler whereby on use of the inhaler the medication is introduced firstly into the spacer before being inhaled by the user. The use of a spacer removes the need to synchronize a user's inhalation with actuation of the inhaler, and also prevents a lot of the drug particles from being deposited on the back of the throat rather than being inhaled into the lungs. Young children in particular have issue synchronizing their breathing with the use of an inhaler, and so the use of a spacer is particularly common with young children.

It is widely acknowledged that spacers are able to improve the delivery of medication into the lungs, and studies have shown that spacers, if used correctly, can provide an equivalent level of bronchodilation as nebulizers. However, this is not reflected in their use and distribution worldwide. A large proportion of low to middle income countries do not have access to commercially produced spacers, mainly as a result of the cost. With increasing urbanization of such low to middle income countries, there has been a simultaneous increase in asthma prevalence, and therefore a need to improve the access to, and efficiency of, asthma medication.

Studies have shown that in children with acute asthma, spacers constructed from sealed cold drinks bottles made from plastic were as effective as conventional spacers. In order to use a plastic cold drinks bottle as a spacer, modification of the bottle is required in order to receive a standard inhaler. In particular, a hole in the base of the bottle needs to be created in order to receive the inhaler, and the hole needs to be skillfully cut order to obtain a good seal between the bottle and the inhaler. This is not always easy, especially when the user is a child.

Furthermore, especially when using such a “home-made” spacer, issues regarding the ability of the user to synchronize their breathing still exist. For example, young children may not be able to coordinate their breathing such that they inhale through their mouth (and therefore inhale the medication from the bottle) and then exhale through their nose to the atmosphere. Users unable to coordinate their breathing in such a manner risk exhaling through their mouth back into the spacer, therefore reducing the efficiency of the medication provision.

The present invention has been devised in order to overcome the problems outlined above.

### SUMMARY OF THE INVENTION

In accordance with a first aspect of the invention, there is provided a closure for a container, comprising: a fixture for attaching the closure to the container; a medicament port for receiving a medicament dispenser and discharging a medicament from the dispenser within an internal volume of the container; an air inlet port for drawing air into the container; a mouthpiece, and; a valve, the valve having an open position which permits air to be drawn from the internal volume of the container into the mouthpiece when a user inhales, and a closed position which seals the mouthpiece from the container and permits air exhaled by the user into the mouthpiece to be vented to atmosphere.

The closure therefore allows the use of a commonly-available glass or plastic drinks bottle to be used as a spacer by attachment of the closure to the container, and overcomes the problems outlined above.

In particular, the fixture for attaching the closure to the container advantageously allows a good air-tight seal to be made between the closure and the drinks bottle such that the efficiency of the drinks bottle as a spacer matches that of a commercially produced spacer. Typically, the fixture comprises a screw thread for communication with a screw thread of a container. For example, it is envisaged that a user may provide a clean and empty plastic or glass drinks bottle for use as a spacer. The user would simply remove the screw cap from the bottle and attach the closure by screwing it onto the screw thread of the bottle. This is particularly advantageous over a scenario as described above where a hole is cut into the base of a bottle in order to receive a standard inhaler. Other means of attaching the closure to the container in order to obtain a good seal are envisaged, for example an O-ring seal.

The closure comprises a valve having an open position which permits air to be drawn from the internal volume of the container into the mouthpiece when a user inhales, and a closed position which seals the mouthpiece from the container and permits air exhaled by the user into the mouthpiece to be vented to atmosphere. Advantageously, this feature of the closure allows a drinks bottle to be used as a spacer with a user simply breathing in and out through their mouth. When inhaling through the mouth, the user receives the desired medication from the bottle due to the open position of the valve, and when exhaling, exhaled air is simply vented to atmosphere rather than re-entering the container and contaminating the medicament. This feature of the closure is particularly beneficial for users who are unable to coordinate their breathing in the desired manner (e.g. children), enabling such a user to inhale and exhale at their leisure while still benefiting from the use of a container acting as a spacer.

Preferably, the valve is actuated by the inhalation and exhalation of the user. For example, the inhalation by the user causes the valve to be in its open position, whereas exhalation by the user causes the valve to be in its closed position. Actuation of the valve in this manner allows for a simple device that is easy to use. However, other means of actuating the valve between the open and closed positions are envisaged as would be known to those skilled in the art, such as electronic, hydraulic or pneumatic actuation.

Typically, the valve comprises a member movable between the open position and the closed position. This member is preferably a disc, wherein the disc is held in the open position by first limit stop and the disc is held in the closed position by a second limit stop. When the valve is actuated by the inhalation and exhalation of the user, when a user inhales, the disc is drawn towards the mouth of the user due to the pressure differential across the disc, and the disc is held in the open position by the first limit stop. Conversely, when the user exhales, the disc is pushed away from the user due to his/her exhalation, and is held in the closed position by a second limit stop. When in the open position, the mouth piece and the container are in fluid communication such that medicament contained within an internal volume of the container may be drawn into the mouthpiece. However, when in the closed position, the mouthpiece and the container are not in fluid communication.

Preferably, the disc has a curved surface structure. This enables smooth and efficient flow of air around the disc when the valve is in the open position.

Although the member movable between the open position and the closed position is preferably a disc, the valve may alternatively be a ball valve wherein the member is a ball, or a diaphragm valve wherein the member is a diaphragm. In the case where the valve is a ball valve, the ball may be biased to a closed position through the use of a spring, and inhalation by the user causes the valve to move to the open position by overcoming the biasing force of the spring.

The closure comprises an air inlet port for drawing air into the container. This means that, when a user of the closure inhales, air is drawn from the atmosphere into the container and into the mouth and lungs of the user. This air flow advantageously means that as much medicament as possible, suspended within the air flow, is provided to the user's lungs at the appropriate speed. Typically, the air inlet port further comprises a tube attached to the air inlet port and extending through the fixture, such that when the closure is attached to the container, the tube extends from the air inlet port into the internal volume of the container. The use of such a tube (for example a plastic straw which can be removably attached to the air inlet port) advantageously enhances the air flow through the container during the inhalation of the user. Such a tube is preferably removably attachable to the air inlet port.

The closure may comprise retaining means configured to hold such a tube in a substantially fixed position relative to the closure and container. The retaining means may comprise a curved surface configured to communicate with a corresponding curved surface of a tube, or a through hole adapted to receive the tube.

Preferably, the closure further comprises at least one vent hole on the distal side of the valve with respect to the container such that when the valve is in the closed position, air exhaled by the user into the mouth piece is vented to atmosphere through the at least one vent hole. As explained above, a particular advantage of the closure is that it may be used by simple inhalation and exhalation through the mouth of a user, thereby negating the requirement of any coordination of breathing through the mouth and nose. When the user exhales, the valve is in the closed position sealing the mouthpiece from the container. Therefore, the exhaled air of the user simply flows through the at least one vent hole of the closure, to atmosphere.

The closure for a container contains a medicament port for receiving a medicament dispenser and discharging a medicament from the dispenser within an internal volume of the container. The medicament is typically dispensed into the closure and subsequently through the fixture into the container. Typically, the medicament port is adapted to communicate with an outlet port of a medicament dispenser. This feature of the closure advantageously allows the user to change the medicament dispenser, for example when it has run out or if a different medicament is required.

Preferably, the medicament port of the closure further comprises a flange such that, when a medicament dispenser in communication with the medicament port is actuated, medicament is jetted into the closure. For example, if the medicament dispenser is an aerosol dispenser, an outlet nozzle of the aerosol container fits into the medicament port of the closure, and the aerosol dispenser is secured by the flange. When the aerosol dispenser is depressed, the flange constrains the nozzle of the dispenser along the axis of compression, allowing the main body of the dispenser to move relative to its nozzle and medicament to be jetted into the closure from the outlet nozzle of the dispenser. The medicament flows from the closure into the container.

Typically, the medicament port of the closure further comprises a nozzle adapted to propel medicament from a medicament dispenser in communication with the medicament port into an internal volume of the container. Preferably, the nozzle comprises an opening having a width less than 0.5 mm, and more preferably has a width between 0.3 and 0.5 mm. Furthermore, the nozzle opening preferably has length less than 0.7 mm. These dimensions advantageously allow for efficient jetting of the medicament into an internal volume of the container such that on use of the closure, the user inhales the desired amount of medicament. The width and axial length may be equal so as to define a circular nozzle opening, or the nozzle opening may be elongate.

The closure may comprise (or allow ready attachment of) a flow meter, configured to measure a metric of the air flow through the closure. Such metrics may include flow rates and/or volumes over time. This might be used to measure the flow of expired and/or inspired air, when attached to the bottle/spacer or when removed from it.

The use of such a flow meter advantageously allows measurement of peak expiratory flow rate, peak inspiratory flow rates, or flow rates throughout the ventilatory cycle such as measure indices of lung function (such as forced vital capacity) or to construct flow-volume loops.

The use of a flow meter allows the user to monitor their lung function and responses to their inhaled drug. The use of such a flow meter advantageously allows a user of the closure or their allocated health professional to keep track of their lung function, inhaler use, response to that inhaler.

The closure may provide direct readout of data. Preferably, however, the flow meter is in wireless communication (using the Internet or other wireless protocols such as Bluetooth) with a computer device, such as a tablet or smart phone such that the measurements obtained by the flow meter can be easily viewed, analysed and stored. However, connection may be direct (e.g. via cable or connector) or by other means as would be known to the skilled person.

Such a flow meter is typically provided in or attachable to the mouthpiece, although it is envisaged that the flow meter may be positioned at any suitable location in the closure.

Preferably, the closure is fabricated from medical grade plastic for hygiene reasons.

The closure may be manufactured by injection moulding. Other methods of manufacture are envisaged however, such as 3D printing. Accordingly, in a second aspect of the present invention, there is provided a computer program product encoding 3D printing instructions to manufacture, using a 3D printer, the closure as described above. It is envisaged that 3D printing of the closure of the present invention will allow for ease of distribution of the closure.

The closure may be 3D printed using a 3D printer to ensure that the finished part has sufficient dimensional integrity, has a smooth enough surface finish and is airtight.

Preferred materials used for the closure include polypropylene, and therefore if the closure is manufactured by injection moulding, food-grade or medical-grade polypropylene may be used. In the case of 3D printing, simulated polypropylene materials such as Stratasys ‘Endur’ material may be used.

According to a third aspect of the invention there is provided a computer program product encoding a computer model of the closure of the invention. A variety of 3D CAD file types may be used for such 3D printing and/or modelling, for example STEP files. The CAD file may be converted into instructions a 3D printer can action.

According to a fourth aspect of the present invention, there is provided a kit comprising the closure as described above, and a medicament dispenser. The kit may also comprise a tube removably attachable to the air inlet port.

Preferably, the medicament port and mouthpiece as described above are removably attachable such they can be fitted together to form the closure. More preferably, the closure comprises three detachable parts: the mouthpiece, medicament port and a disc (where the disc forms part of the valve).

The closure of the present invention overcomes the problems set out in the “Background to the Invention” section and is effective at delivering a medication from a metered dose inhaler, cheap, durable, mass producible and portable.

### BRIEF DESCRIPTION OF THE DRAWINGS

An example of the present invention will now be described in detail with reference to the accompanying drawings, in which:

FIG. 1 shows a side on view of a closure according to an embodiment of the invention attached to a plastic bottle;

FIG. 2 shows a cross-sectional side on view of the closure of FIG. 1 attached to a plastic bottle;

FIG. 3 shows a cross-sectional view of the valve of the closure of FIG. 1 in the open position;

FIG. 4 shows a cross-sectional view of the valve of the closure of FIG. 1 in the closed position;

FIG. 5 shows an end-on cross-sectional view through the closure of FIG. 1, with the disc removed for illustrative purposes;

FIG. 6 shows a side on view of a closure according to a further embodiment of the invention attached to a plastic bottle;

FIG. 7 shows a cross-sectional side on view of the closure of FIG. 6 attached to a plastic bottle, and;

FIG. 8 shows an end-on cross-sectional view through the closure of FIG. 6 with the disc removed for illustrative purposes.

### DETAILED DESCRIPTION

The following describes particular embodiments of the invention. However, it will be appreciated by the skilled person that various alternatives are possible, as discussed above in the summary of invention section.

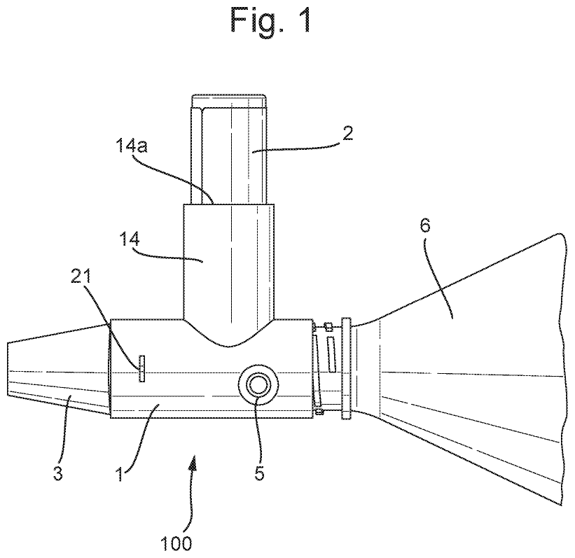

FIG. 1 is a side-on view of a closure 100 of an embodiment of the present invention attached to a commonly-found plastic or glass drinks bottle 6. The closure 100 comprises a mouthpiece 3 having a generally conical shape, a substantially hollow cylindrical main body 1 and a medicament port 14 into which a standard aerosol medicament dispenser 2 is inserted. The mouthpiece 3 is shaped to fit comfortably into the mouth of the user and allow maximum air volume to flow into the user's lungs. The medicament port 14 has a hollow cylindrical form with an opening 14a for receiving and supporting a standard aerosol dispenser, and extends perpendicular to the main body 1. The arrangement of the mouthpiece 3 and main body 1 are such that they are parallel with a long axis of the bottle. This, together with the perpendicular arrangement of the medicament port 14 with respect to a long axis of the main body 1, allows for good ergonomic properties of the closure 100, such that in use it is easy for the user to support the bottle and actuate the aerosol medicament dispenser. The medicament port 14 and main body 1 are typically formed as a unitary member.

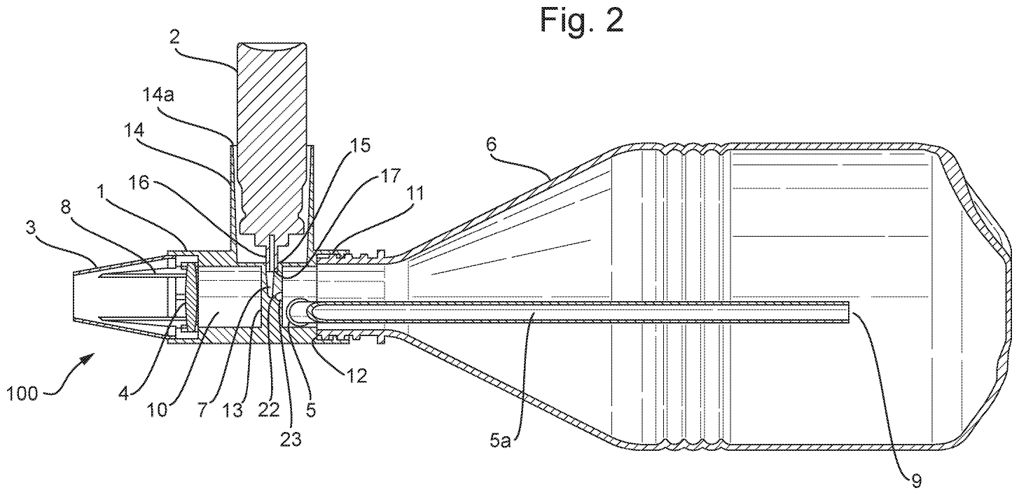

The closure main body 1 is attached to the bottle via a screw thread 11, which is more clearly illustrated in FIG. 2. The main body 1 further comprises vent holes 21 situated on opposing sides of the closure, and an air inlet port 5. These features will be explained in more detail with reference to FIG. 2.

FIG. 2 is a cross-sectional view of the closure 100 attached to a glass or plastic bottle 6. The end of the bottle 6 seals on to a perpendicular face 12 within a main orifice 10 of the main body 1 in order to provide a good seal between the bottle and the closure 100.

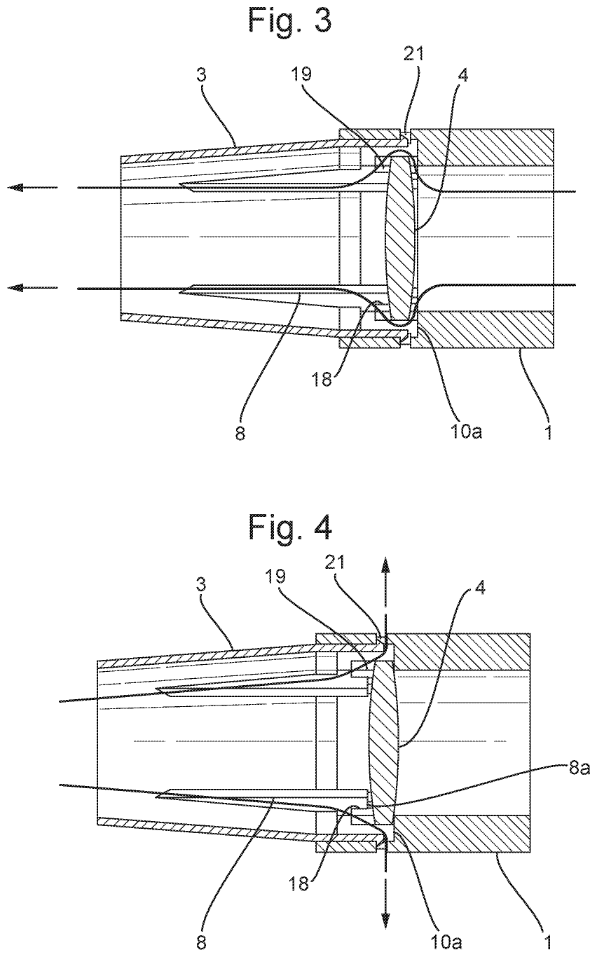

The mouthpiece 3 is removably attachable to the main body 1. The mouthpiece 3 comprises four legs 8 which fit into four corresponding slots 18 of the main body 1. The four slots 18 are located in four corresponding steps 19 of a mouthpiece port of the main body 1. This attachment means is more clearly shown in FIGS. 3 and 4. The cooperation between the legs 8 of the mouthpiece 3 and corresponding slots 18 of the main body 1 ensure a good airtight seal between the mouthpiece and the main body 1.

A disc 4 is situated within the mouthpiece 3 and is allowed to freely move between a mouthpiece end of the main orifice 10 defined by a wall part 10a of the main body 1, and the ends 8a of the legs 8. In this manner, the wall part 10a and the ends 8a of legs 8 act as limit stops for the disc, and the disc, together with the limit stops, act as a valve.

The valve (i.e. the movement of the disc between the first and second limit stops) is actuated by the breathing of the user. When the user inhales through the mouthpiece 3, the disc 4 is sucked against the ends 8a of the legs 8. Here the valve is in the “open” position as air is allowed to flow from the bottle 6 through the main orifice 10 of the main body 1, around the disc 4, into the mouthpiece 3 and subsequently into the lungs of the user. This air flow is illustrated by the arrows in FIG. 3. The disc is substantially cylindrical with rounded end faces to encourage the air flow to divert around the disc and into the mouthpiece. In use, when the user inhales, the pressure differential between the interior of the closure 100 and the surrounding air due to the inhalation causes air to flow through the air inlet port 5, along tube 5a (such as a straw), and flow from the end of the tube into the user's lungs, carrying as much medicament as possible from the bottle suspended in it.

The legs 8 which define the limit stop for the open position of the valve are dimensioned to limit the travel of the disc 4 to a position that allows as much medicament to flow through the valve whilst also keeping the closure as compact as possible in a direction along the main axis of the closure. This allows the closure to be portable and transported in a user's pocket for example.

When the user exhales, the disc 4 is blown against wall part 10a and seals the main orifice 10 of the main body 1 from the user's exhalation. The interior 9 of the bottle is therefore sealed from the user's exhalation, forming a barrier between the user's exhalation and the medicament in the bottle. When the user exhales, the exhalation is instead directed through vent holes 21 in the main body 1 and thus vented to atmosphere. In this position (i.e. the disc being held against the wall part 10a), the disc and valve are in the “closed” position. The air flow during exhalation of a user is illustrated by the arrows in FIG. 4.

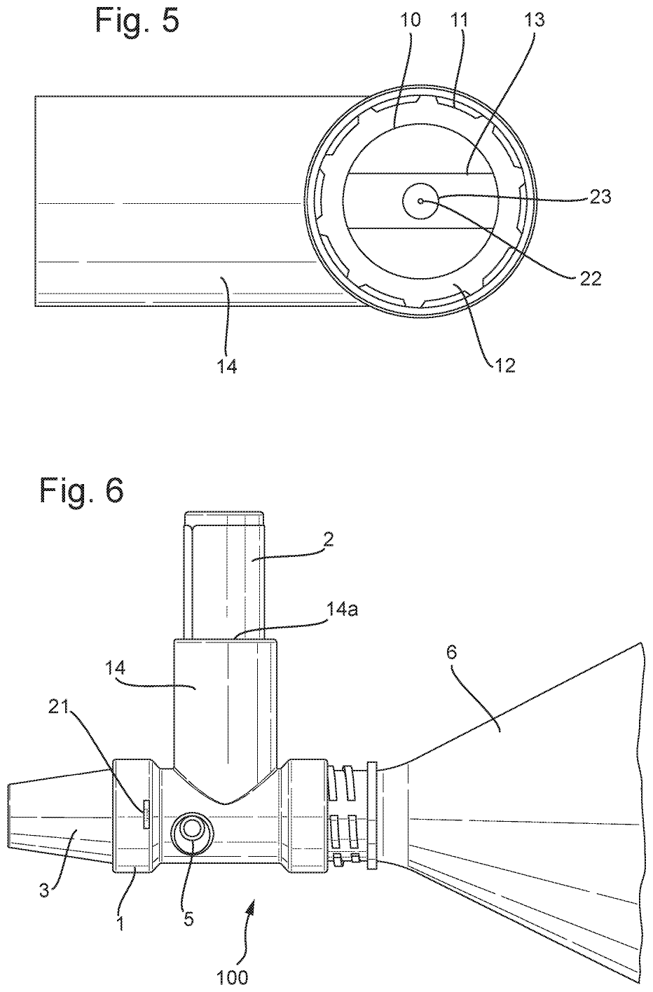

As described above, medicament port 14 is adapted to receive a medicament dispenser 2 such as an IVAX® or Ventolin® Salbutamol dispenser but may be adapted to include others. An outlet nozzle 16 of the medicament dispenser 2 fits tightly into (cooperates with) a receiving port 15 located within a main column 13 situated within the main orifice 10 of the main body 1. Typically the main column 13 is integrally moulded as a part of the main body 1. As shown in FIG. 5 (in which the disc 4 has been removed for illustrative purposes), there is space either side of the main column 13 for air to flow through the main orifice 10 past the main column 13. The main column 13 also comprises an inlet nozzle 7 having an opening 22 which is clearly seen in FIG. 5. The inlet nozzle opening 22 has a width in the range of 0.3 to 0.5 mm, and a length not exceeding 0.75 mm. Here the length is along a direction perpendicular to the long axis of the main body 1, and the width is along a direction parallel to the long axis of the main body 1. The width and axial length may be equal so as to define a circular nozzle opening, or the nozzle opening may be elongate.

The nozzle 7 may optionally comprise a hemispherical diffuser 23 that channels the jet of medicament from the dispenser into the container. The diffuser has a diameter greater than that of the nozzle opening 22, as seen at FIGS. 2 and 5.

The medicament dispenser 2 is supported in a vertical plane within the medicament port 14 by a flange 17, which is an integral part of the receiving port 15. When the medicament dispenser 2 is depressed, for example by a user wishing to inhale a dose of medicament, the flange 17 constrains the outlet nozzle 16 of the dispenser 2 in the vertical plane. Therefore, the housing of the dispenser 2 compresses axially with respect to its outlet nozzle 16, and a metered dose of the user's chosen medicament is dispensed through the outlet nozzle 16 of the dispenser 2, through the receiving port 15, through the inlet nozzle 7 and into the bottle 6. The mechanism for dispensing the correctly metered dose is governed by the manufacturer of the metered dose medicament.

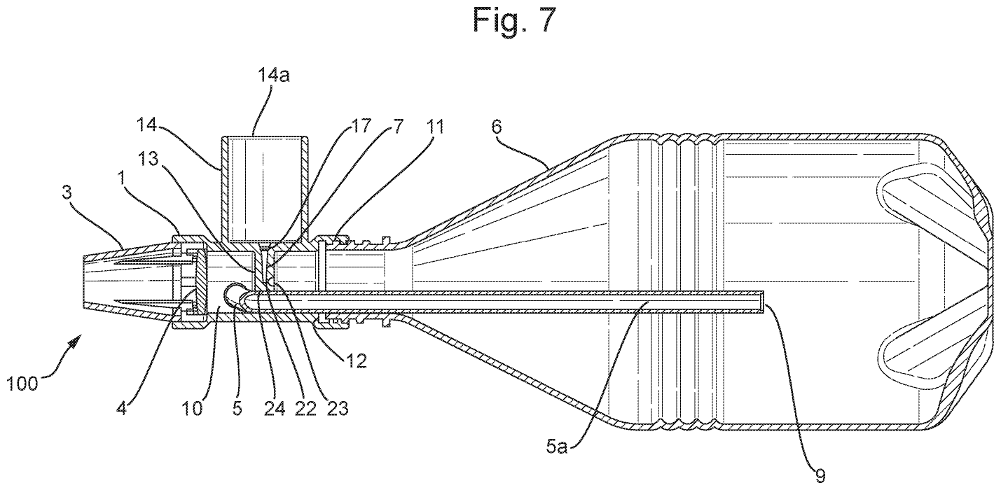

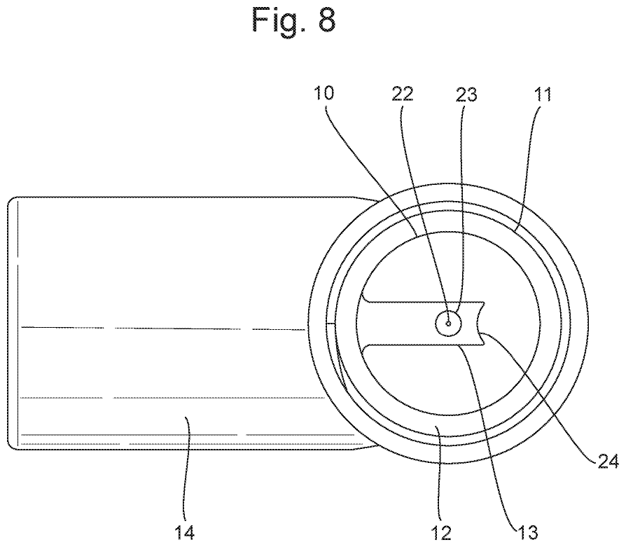

In the above embodiment, the air inlet port 5 is positioned on a distal side of the main column 13 with respect to the mouthpiece 3, and the main column extends between the topmost and bottommost interior surfaces of the main body 1, as seen at FIGS. 2 and 5. In a second embodiment, the air inlet port 5 is positioned on a proximal side of the main column 13 with respect to the mouthpiece 3, and the main column is truncated such that it extends partially from the topmost to the bottommost interior surfaces of the main body. The exposed lower surface of the main column 13 comprises a curved surface 24 configured to retain the tube 5a (where used) in a fixed position relative to the bottle 6. This position of the air inlet port 5 is illustrated in FIGS. 6 and 7 (FIG. 7 illustrates the closure without the medicament dispenser present), and the curved surface 24 of the main column 13 is more clearly seen at FIG. 8.

In other embodiments, the main column may comprise a through hole configured to retain a tube such as a straw. Other forms of retaining means for a tube are envisaged, however.

Through the use of the closure of the present invention, the user is able to inhale and exhale at his/her leisure until an appropriate amount of medicament has been delivered into his/her lungs, without having to worry about coordination of their breathing.

Optionally, the mouthpiece 3 of the closure of the present invention may comprise a flow meter (not shown) configured to measure a metric relating to the user's lung function based on the flow of air through the mouthpiece. The flow meter may be positioned in other parts of the closure, however. Such a measurement may be a peak expiratory flow measurement for example. The flow meter may be configured to display its measurements on the closure 100 itself, for example through a readable digital or analogue scale, or other display means. Alternatively or in addition, the flow meter may be in wireless communication with a computer device by means of the Internet or other wireless protocols such as Bluetooth.

The closure of the present invention is manufactured using 3D printing of a suitable CAD design file, although other methods of manufacture are possible such as injection moulding, as will be appreciated by those skilled in the art. The closure is manufactured from medical grade plastic. In particular, the closure of the described embodiment comprises four detachable parts: the main body and medicament port, the disc, the mouthpiece and the tube.

1-29. (canceled)

30. A closure for a container, comprising:
a fixture for attaching the closure to the container;
a medicament port for receiving a medicament dispenser and discharging a medicament from the dispenser within an internal volume of the container;
an air inlet port for drawing air into the container;
a mouthpiece, and;
a valve, the valve having an open position which permits air to be drawn from the internal volume of the container into the mouthpiece when a user inhales, and a closed position which seals the mouthpiece from the container and permits air exhaled by the user into the mouthpiece to be vented to atmosphere.

31. The closure of claim 30, wherein the valve is actuated by the inhalation and exhalation of the user.

32. The closure of claim 30, wherein the valve comprises a member moveable between the open position and the closed position.

33. The closure of claim 32, wherein the member is a disc, and wherein the disc is held in the open position by a first limit stop and the disc is held in the closed position by a second limit stop, and wherein the disc has a curved surface structure.

34. The closure of claim 32, wherein the member is a ball.

35. The closure of claim 32, wherein the member is a diaphragm.

36. The closure of claim 30, further comprising a tube attached to the air inlet port and extending through the fixture, such that when the closure is attached to the container, the tube extends from the air inlet port into the internal volume of the container.

37. The closure of claim 36, wherein the closure comprises a retainer configured to hold the tube in a substantially fixed position.

38. The closure of claim 37, wherein the retainer comprises a curved surface configured to communicate with a corresponding curved surface of the tube.

39. The closure of claim 36, wherein the tube is removably attachable to the air inlet port.

40. The closure of claim 30, further comprising at least one vent hole on the distal side of the valve with respect to the container such that when the valve is in the closed position, air exhaled by the user into the mouthpiece is vented to atmosphere through the at least one vent hole.

41. The closure of claim 30, wherein the medicament port is adapted to communicate with an outlet port of a medicament dispenser, and wherein the medicament port further comprises a flange such that, when a medicament dispenser in communication with the medicament port is actuated, medicament is jetted into the closure.

42. The closure of claim 41, wherein the medicament port further comprises a nozzle adapted to propel medicament from a medicament dispenser in communication with the medicament port into an internal volume of the container.

43. The closure of claim 30, wherein the fixture comprises a screw thread for communication with a screw thread of a container.

44. The closure of claim 30, wherein the closure is fabricated from medical grade plastic.

45. The closure of claim 30, further comprising a flow meter configured to measure a metric of the air flow through the closure.

46. The closure of claim 45, wherein the flow meter is in wireless communication with a computer device.

47. The closure of claim 30, wherein the closure is manufactured by injection moulding or 3D printing.

48. A kit comprising;
the closure of claim 30, and;
a medicament dispenser.

49. An assembly comprising a medicament port and a mouthpiece as parts which can be fitted together to form the closure of claim 30.
